(19) United States
(12) Patent Application Publication
(10) Pub. No.: US 2016/0375061 A1
(43) Pub. Date: Dec. 29, 2016

(54) COMPOSITION INVOLVED IN REGULATING ENERGY CYCLE, INFLAMMATION AND INSULIN RESISTANCE DYSFUNCTIONS, AND USE THEREOF PARTICULARLY IN CARDIOMETABOLIC DISEASES

(71) Applicant: INTERNATIONAL NUTRITION RESEARCH COMPANY, Luxembourg (LU)

(72) Inventor: Claude VINCENT, Bordeaux (FR)

(21) Appl. No.: 14/902,518

(22) PCT Filed: Jul. 2, 2014
(86) PCT No.: PCT/EP2014/064110

§ 371 Date: Dec. 31, 2015

(30) Foreign Application Priority Data
Jul. 2, 2013 (FR) 1356444
Jul. 2, 2013 (FR) 1356446
Jul. 2, 2013 (FR) 1356448
Jul. 2, 2013 (FR) 1356450

(51) Int. Cl. A61K 35/20; A61K 31/4415; A61K 31/355; A61K 31/4375; A61K 31/59; A61K 31/202; A61K 38/44; A61K 38/06; A61K 33/06; A61K 38/19; A61K 31/198; A61K 31/405; A61K 31/155; A61K 33/30; A61K 33/04; A61K 31/366; A61K 31/401; A61K 9/00; A61K 38/38

(52) U.S. Cl. CPC A61K 35/20; A61K 38/38; A61K 31/4415; A61K 31/355; A61K 31/4375; A61K 31/59; A61K 31/202; A61K 38/446; A61K 38/06; A61K 33/06; A61K 38/193; A61K 31/198; A61K 31/405; A61K 31/155; A61K 33/30; A61K 33/04; A61K 31/366; A61K 31/401; A61K 9/0053; C12Y 115/01001

(57) ABSTRACT

A composition suitable for use as a health product, including a mixture of active ingredients made up of at least: a lactoserum hydrolysate with a molecular weight between 200 and 5000 daltons, alpha-lactalbumin, at least one AMPK, MAPK, PPAR and/or SIRT1 cycle regulator, and milk calcium. The composition is particularly useful as a medical nutrition product or orally administered drug, to prevent and/or combat various diseases.

### FIELD OF INVENTION

This invention relates to a specific composition and its use as a health product, in particular for the prevention of cardiometabolic risk, for the prevention and treatment of cardiometabolic diseases, and for the prevention and the treatment of infertility and/or subfertility of overweight persons.

### BACKGROUND OF INVENTION

The cardiometabolic risk or metabolic syndrome refers to the presence in an individual of multiple biological and clinical signs that demonstrate the beginning of type 2 diabetes and cardiac diseases, without, however, these signs being necessarily felt by the affected person. It is characterized by the association of multiple manifestations, in particular a large abdominal circumference and/or a high level of triglycerides in the blood and/or an increase in glucose intolerance (high glycemia) and/or insulin resistance and/or arterial hypertension and/or a reduced HDL level and/or an increase in fibrinogen and/or inflammatory adipocytokines: TNFα (“Tumor Necrosis Factor α”), IL6 (interleukin 6), PAI-1 (“Plasminogen activator inhibitor-1”), ultrasensitive CRP (“C-reactive protein”), which bring about a reduction of adiponectin and serotonin, which are central hormones in this type of impairment.

The risk of cardiovascular and metabolic anomalies is correlated with excess weight and, in the majority of cases, with a poor lifestyle. Nevertheless, not all overweight conditions necessarily induce life-threatening health risks. Actually, cardiometabolic problems are linked to visceral fat, i.e., deep fat around the waistline and deep subcutaneous fat. The subcutaneous fat of the first two layers, although it is unsightly, does not pose a health problem in itself. Visceral fat and fat of the deep subcutaneous layer contain very active macrophages from a hormonal standpoint, which lead to impairments of the liver, the pancreas and the energy-regulating system at the cellular level, and in terms of the brain and the intestine, as well as numerous deficiencies caused by chronic and minor inflammation of the body, which is the central problem of this type of chronic disease. This chronic inflammation is responsible for the formation of fatty plaque in the vessels, which is deposited on the walls (angina pectoris and arteritis of the lower limbs) and ends up rupturing (heart attack and CVA [cerebrovascular accident]) owing to the action of one of the adipocytokines of the inflammation of the us-CRP (ultra-sensitive C reactive protein). In addition, because of the formation of their waste, free radicals hamper the exchanges at the cellular level, causing glucose intolerance, insulin resistance, and then diabetes by a matrix remodeling of the cell.

Furthermore, the liver is particularly sensitive to the metabolic syndrome since it is the first site of deposition of triglycerides, and with the increase in storage, hepatic steatosis occurs that can evolve into NAFLD (“Nonalcoholic Fatty Liver Disease,” non-alcoholic hepatic steatosis), NASH (“Nonalcoholic Steatohepatitis”), with fibrosis that is more or less significant and that develops into cirrhosis and cancer.

It is therefore necessary to find solutions that make it possible to prevent cardiometabolic risk, in particular treatments that make it possible to prevent hepatic steatosis and to block the transition from prediabetes to type 2 diabetes by lowering the glycemia and by normalizing the risk factors such as hyperlipidemia, hypertension, hyperglycemia and hyperinsulinemia, for delaying or stopping cardiovascular diseases.

The existing solutions for preventing cardiometabolic risk are in general limited to the application of dietary regimens. However, none of the regimens proposed since the development of cardiometabolic risk in the 1990s, be they balanced, hyperproteinated, hyperglucidic, hyperlipidic, very restrictive or unbalanced, have solved the specific problems of impairments and pathological particularities of this syndrome. Very restrictive protein diets even aggravate metabolic impairments and cause rebound effects.

Furthermore, the existing medications that are used act on one of the consequences of the metabolic syndrome, such as hyperglycemia, hypertension, hypertriglyceridemia, or hypercholesterolemia, but not on the entire metabolic syndrome and even sometimes aggravate another component of the metabolic syndrome (such as, for example, the current statin-based products that lead to diabetogenesis).

More recently, dietary products intended to reduce deep subcutaneous and visceral fat have been developed and used for the prevention of cardiometabolic risk.

### SUMMARY OF INVENTION

The objective of this invention is to propose a different solution that acts on specific pathways of the metabolic syndrome, simple to use and effective, which is capable of preventing cardiometabolic risk, preventing hepatic steatosis, and treating prediabetes, but also of treating cardiovascular diseases in general and preventing and treating infertility and subfertility in men and women.

To respond to this, this invention proposes to act both on the adiponectin level and the serotonin level and to provide small active biopeptides and amino acids, which act at the cellular level on the cycle of the energy of AMP (adenosine monophosphate) and ATP (adenosine triphosphate) through all of the cycles of AMPK (“AMP-activated protein kinase”), MAPK (“mitogen-activated protein kinase”), PPAR (“peroxisome proliferator activated receptors”) and SIRT1 (sirtuin 1), and on the cycles of inflammation and insulin resistance.

For this purpose, the object of the invention is a composition that is suitable for use as a health product with oral administration, in particular as a medical nutrition product or medication, comprising a mixture of active ingredients that consist of at least:
A lactoserum hydrolyzate with a molecular weight of between 200 and 5,000 daltons,
Alpha-lactalbumin,
At least one regulator of the cycles of AMPK and/or MAPK and/or PPARα and γ and/or SIRT1, in particular a mimetic agent or agonist of thiazolinediones, selected from among the family of tocopherols and vitamin B6 and leukine and the family of branched amino acids, and
Milk calcium,
And optionally at least one pharmaceutical active ingredient that regulates the cycles of AMPK and/or MAPK and/or PPARα and γ and/or SIRT1, selected from among metformin and statins.

Advantageously, such a composition makes it possible to reduce visceral fat, hepatic fat, and deep subcutaneous fat by making possible in particular an increase of the adiponectin and serotonin levels. It can be used as a health product in particular in the prevention of cardiometabolic risk and/or hepatic steatosis and/or in the prevention and/or treatment of prediabetes (by preventing the transition from prediabetes into type 2 diabetes), type 2 diabetes, cardiovascular diseases and/or infertility and/or subfertility in overweight men or women. It is involved in the regulation of the impairment of cycles of energy, and cycles of inflammation, and insulin resistance.

Preferably, it is used to supplement a change in lifestyle involving especially a particular diet, limiting calorie intake, physical activity, management of chronic stress, and management of a state of anxiety requiring therapeutic education of the patient.

### DETAILED DESCRIPTION OF THE INVENTION

The invention is now described in detail.

The object of the invention is therefore a composition that is suitable for use as a health product with oral administration, comprising a mixture of active ingredients that consists of at least:
A lactoserum hydrolyzate with a molecular weight of between 200 and 5,000 daltons,
Alpha-lactalbumin,
At least one regulator of the cycles of AMPK and/or MAPK and/or PPARα and γ and/or SIRT1, selected from among the family of tocopherols, vitamin B6, leukine, and the family of branched amino acids, and
Milk calcium.

According to a variant, the composition can also contain in addition at least one pharmaceutical active ingredient that regulates cycles of AMPK and/or MAPK and/or PPARα and γ and/or SIRT1, selected from among metformin and statins.

In terms of the invention, health product is defined as any product that acts to benefit human health, in particular a medication or a medical nutrition product.

In terms of the invention, medical nutrition product is defined as a food with a therapeutic purpose of prevention or treatment, used by itself or combined with other therapies.

The composition according to the invention is particularly suitable for persons who desire to lose visceral fat, hepatic fat and/or subcutaneous fat in particular for medical reasons of primary or secondary or tertiary prevention of cardiovascular diseases, hepatic steatosis, prediabetes, diabetes and/or infertility and/or subfertility.

Prediabetes is defined as type 2 prediabetes, and these terms are used interchangeably to describe this invention. Likewise, diabetes is defined as type 2 diabetes, and these terms are used interchangeably to describe this invention.

Cardiometabolic risk is defined as a syndrome that combines at least 3 risk factors such as the waistline, glycemia and arterial tension, HDL cholesterol, nicotine addiction, and heredity. This metabolic syndrome leads to multiple pathologies that are in particular: type 2 diabetes, atherosclerosis, ischemic coronaropathy, cerebrovascular dementia, cerebrovascular accident, obliterating arteriopathy of the lower limbs, metabolic infertility, and hepatic diseases: steatosis, fibrosis, cirrhosis.

In terms of the invention, lactoserum hydrolyzate is defined as any molecule or mixture of molecules obtained from lactoserum, in particular lactoserum hydrolyzed by a process that comprises a stage of chemical hydrolysis or enzymatic hydrolysis.

In a particularly suitable way, it is a matter of a peptidic hydrolyzate. It preferably comprises at least 75% proteins or peptides, by weight of the hydrolyzate.

The lactoserum hydrolyzate has a molecular weight of between 200 and 5,000 daltons. Preferably, at least 55% of the total of the molecules of the lactoserum hydrolyzate has a molecular weight that is less than or equal to 1,000 daltons.

It makes sense to promote the smallest size possible of the molecules because it makes it possible to open up the active biopeptides of the lactoserum that are prisoners of macropeptides. This small size makes it possible for the molecules to pass through the digestive tract quickly, between 15 and 30 minutes, in an intact manner without being enveloped in a mass that will be randomly cut up by digestive enzymes.

The lactoserum hydrolyzate essentially consists of small peptides, in particular bi-or tri-peptides.

The size of the molecules in the hydrolyzate can be modulated by microfiltration.

Very preferably, the hydrolyzate is obtained with hydrolysis whose ratio is between 15 and 35%. The ratio cannot be greater, for the moment, than 35%, because above 35%, the bitterness is so pronounced that it cannot be controlled and in addition the composition would have more free amino acids than active biopeptides, which could cause the treatment to lose effectiveness and control.

Alpha-lactalbumin is a nutritional active ingredient that composes lactoserum that is particularly rich in tryptophan and branched amino acids.

Milk calcium is a calcium that is extracted from milk and that preserves a higher therapeutic action than synthetic calcium because of the residual presence of small bioactive peptides.

Regulator of the cycles of AMPK and/or MAPK and/or PPARα and γ and/or SIRT1 is defined as any molecule or mixture of molecules acting on at least one of the cycles of AMPK and/or MAPK and/or PPARα and γ and/or SIRT1. It may involve:
A nutritional active ingredient such as the tocopherols, vitamin B6, leukine, or branched amino acids: the composition can contain one or more tocopherol(s), or vitamin B6, or leukine, or one or more branched amino acid(s), or a mixture of at least two of these molecules,
A pharmaceutical active ingredient such as metformin and statins: the composition can comprise metformin, or one or more statins, or a mixture of metformin and statin(s).

The regulator of the cycles of AMPK and/or MAPK and/or PPARα and γ and/or SIRT1 is preferably a mimetic agent or an agonist of thiazolinediones.

Mimetic agent of the thiazolinediones is defined as a molecule or mixture of molecules that integrally copies the mode of action of thiazolinediones because of the similar chemical structure thereof, but as much as possible without having the drawbacks thereof.

Agonist of thiazolinediones is defined as a molecule or mixture of molecules that acts in the same manner as the thiazolinediones, but as much as possible without having the drawbacks thereof.

The thiazolinediones have the advantage of acting in particular as a regulator with a powerful action of the cycles of PPARγ and AMPK and indirectly on SIRT1. This action mimics a limiting of calorie intake accompanied by physical activity. However, the associated side effects, such as weight gain, hepatitis, salt retention, and an increase in cardiac risk due to the increase in foam cells, led to withdrawal of the approvals to market thiazolindediones in numerous countries.

According to the invention, the mimetic agent or the agonist of thiazolinediones is selected from among the family of tocopherols or vitamin B6 or leukine or from among the family of branched amino acids. If tocopherol is involved, it should preferably be a mixture of α and γ tocopherol, even more preferably in proportions of 75% for γ and 25% for α.

The composition according to the invention can optionally contain one or more pharmaceutical active ingredient(s) activating cycles of AMPK and/or MAPK and/or PPARα and γ and/or SIRT1, selected from among metformin and statins, in particular simvastatin because of its workability, but also atorvastatin, pravastatin, rosuvastatin, fluvastatin, or lovastatin or a mixture of at least two of these statins. The quantity of pharmaceutical active ingredient(s) regulating cycles of AMPK and/or MAPK and/or PPARα and γ and/or SIRT1 is 200 to 700 mg for metformin and 2.5 to 10 mg for statins, for between 20 and 40 g of composition without vehicles.

The different components of the composition act in synergy for a very effective action.

The composition according to the invention is in particular capable of acting on the adiponectin level.

A diponectin is a molecule that is produced in particular by the adipocytes of the visceral adipose tissue. On the peripheral level, it acts via two receptors, through which it regulates the cycles of AMPK, MAPK, PPARα, PPARγ, and SIRT1 and inhibits the signal NFkB.

These effects intersect since the secretion of adiponectin is itself dependent upon multiple factors, including AMPK, MAPK, PPAR and SIRT1: a dephosphorylation of AMPK, MAPK, PPAR and/or SIRT1 brings about a reduction of the secretion of adiponectin. The role of adiponectin is multiple, since in particular it is capable of:
Eliminating excess ROS (reactive oxidized radicals) and RNS (reactive nitrogen-containing radicals),
Increasing nitric oxide NO through AMPK,
Decreasing the expression of LPS (lipopolysaccharide) that activates the macrophages of visceral adipose tissue,
Decreasing inflammation in particular of TNFα by the modulation of the signal NFkB,
Remediating the consequences of the oxidation and reducing the effects of ischemia on damaged tissue (in particular in myocardial infarction),
Regulating the calcification of arteries caused by TNFα through AMPK,
Correlating positively with HDL cholesterol (“High Density Lipoprotein”) but negatively with triglycerides,
Regulating AMPK and MAPK and therefore the AMP/ATP ratio,
Acting on a decrease in hepatic steatosis,
Decreasing in the vessels the platelet adhesion and their migration and slowing down the formation of foam cells,
Increasing the use of glucose and the β-oxidation of the fatty acids in the muscle, and therefore preventing the diabetic state,
Decreasing glycogenesis in the liver and increasing the β-oxidation of fatty acids,
Linking directly with the microbiota for its HMW (“high molecular weight”) form.

However, the adiponectin level is very low in persons having a cardiometabolic risk, but also in obese individuals with insulin resistance, diabetics, persons with lipodystrophy, persons with hepatic steatosis, NAFLD, NASH, persons in hypertension and persons with a coronopathy. Adiponectin is deficient in particular because of the increase of inflammatory adipocytokines, in particular TNFα, which act negatively through the transcription, the translation, and the turnover of AMPK, MAPK, PPAR and SIRT1, causing their reduction by dephosphorylation and therefore a decrease in the adiponectin secretion.

The composition according to the invention makes it possible to increase the level of circulating adiponectin and/or to regulate its secretion over the long term.

In particular, the presence of at least one regulator of the cycles of AMPK and/or MAPK and/or PPARα and γ and/or SIRT1, in particular a mimetic agent or agonist of thiazolinediones, makes it possible to activate AMPK and/or MAPK and/or PPARα and γ and/or SIRT1 and therefore to increase the adiponectin level.

The milk calcium also makes it possible to increase the level of adiponectin, in particular HMW adiponectine.

According to another aspect, the composition according to the invention is capable of acting on the serotonin level.

Serotonin is a hormone that acts in particular on mood and depression, stress, sleep, food intake, and eating habits that deviate like bulimias. It is found essentially in the intestine at the level of chromaffin cells, but also in the platelets and in the brain.

The precursor of serotonin is tryptophan that is converted into 5HTP and then into 5HT, serotonin. Its metabolites are 5-HIAA (5-hydroxyindoleacetic acid), 5-HTOL (5-hydroxy-tryptophol), and its derivatives N-acetyl-5HT and melatonin.

Only the freely-circulating tryptophan, which represents only 1% tryptophan, is capable of passing through the encephalic barrier. In addition, to pass through the encephalic barrier, tryptophan is in competition with the branched amino acids and aromatic compounds, i.e., the neutral compounds, with the physiological threshold of the ratio of tryptophan to these neutral amino acids being 6%. In terms of the brain, the half-life of serotonin is several minutes. It is therefore necessary to have a large quantity of plasmatic tryptophan for making possible a regular passage of the barrier and to ensure that the brain has the necessary quantity of serotonin. In terms of the intestine, the serotonin is abundant (80% of the total production) and activates the protein GLP1 that communicates directly with the brain for initiating the feeling of fullness. There are thus two systems that determine fullness and satisfaction in terms of the brain.

The metabolic syndrome and excess abdominal weight bring about a serotonin deficiency.

The inflammation of adipocytokines brings about the degradation of serotonin into 5HIAA in terms of the liver. Actually, the inflammation mainly afflicts the kynurenic pathway.

The composition according to the invention makes it possible to increase the serotonin level in particular using tryptophan that is provided in particular by the lactoserum hydrolyzate and alpha-lactalbumin, but also by the reduction of inflammation. Tryptophan should preferably represent between 6 and 9%, even more preferably approximately 7%, by weight of the neutral amino acids that are present in the composition. This particular proportion makes it possible to ensure that a suitable quantity of tryptophan can pass through the encephalic barrier to be converted into serotonin. The daily supply of tryptophan by the composition according to the invention is preferably, for the persons in a metabolic syndrome or in a stage of prediabetes, at least 0.5 g, preferably 0.5 to 0.8 g. This increase in the serotonin level, in addition to its direct effect in particular on the fullness feeling, also plays a part to promote an increase in the adiponectin level. Actually, the metabolites of serotonin act as agonists of PPARγ and therefore stimulate the secretion of adiponectin.

Furthermore, results of tests conducted with the lactoserum hydrolyzate according to the invention on white adipose tissue have shown that the cycle of the energy was activated by increased consumption of oxygen by 63% and that the lipolysis of the adipocytes was accelerated by the release of glycerol (20%), and finally that the conversion of preadipocytes into adipocytes was slowed down by the modulation of MCP1.

Finally, the composition according to the invention makes it possible to provide a large quantity of quality amino acids, in particular leukine and isoleukine, in addition to the tryptophan already mentioned.

Leukine is in particular capable of acting on SIRT1, AMPK and PPARγ, and therefore makes possible an increase in adiponectin. In addition, hydroxybutyrate, active metabolite of leukine, regulates the β-oxidation of the lipids through AMPK and SIRT1. According to the invention, leukine can be obtained from the lactoserum hydrolyzate and from alpha-lactalbumin, and it can also be used by itself, as a regulator of the cycles of AMPK and/or MAPK and/or PPARα and γ and/or SIRT1.

Isoleukine induces a reduction in glycemia by increasing the use of glucose through AMPK in particular.

In the composition, the ratio of the weight of the total amino acids/leukine of the composition is preferably between 10 and 15%, the ratio by weight of neutral amino acids/leukine of the composition is preferably between 30 and 40%, and the ratio by weight of branched acids/leukine of the composition is preferably between 45 and 55%. The daily supply of leukine by the composition according to the invention is preferably at least 5 g.

Furthermore, in the composition, the ratio by weight of total amino acids/isoleukine of the composition is preferably between 5 and 10%, the ratio by weight of neutral amino acids/isoleukine of the composition is preferably between 15 and 25%, and the ratio by weight of branched acids/isoleukine of the composition is preferably between 20 and 30%. The daily supply of isoleukine by the composition according to the invention is preferably at least 2.5 g.

Tryptophan, leukine, and isoleukine are contained in the particular lactoserum hydrolyzate that is used and that is in alpha-lactalbumin. The selection of these two compounds is essential for the effect of the composition.

First of all, according to the invention, the lactoserum milk proteins are more suitable than the vegetable proteins, which have fewer branched amino acids, or than the biotechnology fermentation amino acids, which have a lower activity than that of the lactoserum amino acids because of the absence of active biopeptides. Likewise, the form of the lactoserum is important because the digestibility and therefore the availability of the amino acids is different and acts on the chronobiology of the intake depending on whether the concentrate, the isolate, or the hydrolyzate is used. The best results for meeting the objective of the invention are obtained with a hydrolyzate that contains small molecules, in particular bi- and tri-peptides that highlight the biopeptides of the lactoserum. The molecules of the hydrolyzate should have a size of between 200 and 5,000 daltons that represent the optimal presence of lactoserum biopeptides that are opened up from the macropeptides. For further optimized effectiveness, at least 55% of the total of the lactoserum hydrolyzate molecules has a molecular weight that is less than or equal to 1,000 daltons, which corresponds to a peptide of 6 amino acids maximum. To obtain molecules that are so small, it is necessary to resort to hydrolysis of the lactoserum with a degree of hydrolysis of between 15% and 35%.

The presence of alpha-lactalbumin is necessary in the composition according to the invention because the use of a lactoserum hydrolyzate by itself, even one of excellent quality, is not sufficient for providing the necessary quantities and proportions of certain amino acids, in particular tryptophan, leukine, and isoleukine. In addition, the alpha-lactalbumin like the milk calcium make it possible to minimize the bitterness of the lactoserum, in particular in the case of hydrolysis with a ratio of between 15% and 35%, which increases this bitterness.

Alpha-lactalbumin is very preferably present in a proportion that is less than or equal to 50% by weight relative to the lactoserum. The ratio by weight of proteins between the alpha-lactalbumin and the hydrolyzate is between 50/50 and 30/70.

The mixture of lactoserum hydrolyzate and alpha-lactalbumin of the composition according to the invention should therefore preferably contain at least tryptophan, leukine, and isoleukine. Even more preferably, it also contains glutamine and arginine.

The presence of glutamine or glutamic acid makes it possible to reduce the hyperpermeability of the intestinal barrier. The hyperpermeability of the intestinal barrier, through the passage of LPS, is involved in the activation of the signal NFkB and therefore in the reduction of adiponectin and primarily the increase in inflammatory adipocytokines such as IL6 and TNFα. The action of glutamine or glutamic acid is reinforced by the presence of leukine and arginine. Preferably, glutamine and/or glutamic acid represent(s) more than 150% of the weight of the leukine that is present in the composition. Likewise, if arginine is also present, the proportion by weight of glutamine and/or glutamic acid is preferably at least 7 times that of arginine.

The composition can also contain other amino acids such as valine, phenylalanine, or tyrosine in particular.

In addition to the mixture of hydrolyzate, alpha-lactalbumin, one or more regulator(s) of the cycles of AMPK and/or MAPK and/or PPARα and γ and/or SIRT1, and milk calcium, the mixture of active ingredients of the composition according to the invention can comprise one or more freely-added elements that are selected in particular from among vitamin B9 and vitamin D.

Likewise, the composition can contain essential fatty acids, in particular omega-3 fatty acids. Preferably, it involves omega-3 fatty acids of vegetable origin ALA (alpha-linolenic acid), with a high proportion of EPA (eicosapentanoic acid) or else directly purified EPA.

The optional presence of metformin has the advantage of acting through AMPK for inhibiting hepatic glycogenesis and improves the endothelial impairment, but it is associated with numerous side effects that disrupt the treatment, such as nausea and vomiting and in the worst cases lactic acidosis. These intolerances can be caused in particular by renal insufficiency but also by the mutation of the gene transporter OCT1 that causes a saturation of metformin with concentrations of 30±19 ng/ml whereas the daily doses of medication were normal from 1 to 3 g/day. The presence of other components of the composition according to the invention makes it possible to be able to lower the doses for activating cycles of AMPK and/or MAPK and/or PPARα and γ and/or SIRT1, in particular metformin, while preserving the same activity but without side effects. The daily dose of metformin in the composition according to the invention is less than or equal to 1 g, preferably between 500 mg and 1 g.

According to a variant, the composition can also contain additional components that are known for improving the adaptation of the cycles of AMPK, MAPK, PPAR and SIRT1, such as zinc, chromium, curcumin, SOD [superoxide dismutase], catalase, glutathione, resveratrol, and/or anthocyanins. Curcumin, at a low dose so as not to reach its pro-oxidizing threshold, makes it possible to reduce the hyperpermeability of the intestinal barrier like glutamine. The SOD, the catalase, and the glutathione, by the powerful anti-oxidizing role thereof, attack the ROS and indirectly modulate the signal NFkB of the inflammation and therefore participate in an increase in the secretion of adiponectin. The SOD also acts on the prevention of the β-oxidation of LDL. Resveratrol, anthocyanins, and curcumin act at the PPAR and AMPK level.

The various components of the composition according to the invention act in synergy for obtaining surprising effects that are suitable in particular for the prevention of cardiometabolic risk and hepatic steatosis and for the treatment of the prediabetes state.

According to a preferred embodiment, the mixture of active ingredients of the composition according to the invention comprises at least:
Between 40 and 60% lactoserum hydrolyzate with a molecular weight of between 200 and 5,000 daltons,
Between 38 and 55% alpha-lactalbumin,
Between 5 and 30 mg, preferably between 5 and 15 mg, of at least one nutritional regulator of the cycles of AMPK and/or MAPK and/or PPARα and γ and/or SIRT1 selected from among the family of tocopherols, vitamin B6, leukine, the family of branched amino acids, for between 25 and 40 g of composition without vehicles,
Optionally one or more pharmaceutical active ingredient(s) regulating cycles of AMPK and/or MAPK and/or PPARα and γ and/or SIRT1 selected from among metformin and statins,
Between 1 and 3% milk calcium,
   with the percentages being given by weight of dry material of all of the active ingredients that are present in the composition (except for possible vehicles).

The composition can also contain freely-added elements, such as vitamins and minerals, which are added to the native components of the hydrolyzate, alpha-lactalbumin, the regulator(s) of the cycles of AMPK and/or MAPK and/or PPARα and γ and/or SIRT1.

The composition according to the invention preferably consists of at least:
1 to 3% tryptophan,
12 to 20% branched amino acids,
6 to 10% aromatic amino acids,
10 and 20% glutamine or glutamic acid,
1.6 to 3% arginine,
1.2 to 3% milk calcium,
0.2 to 1% EPA,
1 to 2 mg of vitamin B6 per 30 g of composition without vehicles,
1 to 3 μg of vitamin D per 30 g of composition without vehicles,
75 to 150 μg of vitamin B9 per 30 g of composition without vehicles,
5 to 15 mg of vitamin E per 30 g of composition without vehicles,
   with the percentages being given by weight of dry material of all of the active ingredients that are present in the composition (outside of possible vehicles), a portion of the components originating from the hydrolyzate, alpha-lactalbumin, regulator(s) of the cycles of AMPK and/or MAPK and/or PPARα and γ and/or SIRT1 and from milk calcium, and the remainder being added freely in the form of vitamins, minerals, and fatty acids.

According to another preferred embodiment, the mixture of active ingredients of the composition according to the invention comprises at least:
Between 40 and 60% lactoserum hydrolyzate with a molecular weight of between 200 and 5,000 daltons,
Between 38 and 55% alpha-lactalbumin,
Between 250 and 500 mg of pharmaceutical active ingredient(s) activating cycles of AMPK and/or MAPK and/or PPARα and γ and/or SIRT1 selected from among metformin and statins, for between 20 and 40 g of composition without vehicles, and
Between 5 and 30 mg, preferably between 5 and 15 mg, of at least one nutritional regulator of the cycles of AMPK and/or MAPK and/or PPARα and γ and/or SIRT1 selected from among the family of tocopherols, vitamin B6, leukine, the family of branched amino acids, for between 20 and 40 g of composition without vehicles, and
Between 1 and 3% milk calcium,
   with the percentages being given by weight of dry material of all of the active ingredients that are present in the composition (except for possible vehicles).

The composition can also contain freely-added elements, such as vitamins and minerals, which were just added to the native components of the hydrolyzate, alpha-lactalbumin, and the regulator(s) of the cycles of AMPK and/or MAPK and/or PPARα and γ and/or SIRT1.

The composition according to the invention can consist of at least:
1 to 3% tryptophan,
12 to 20% branched amino acids,
6 to 10% aromatic amino acids,
10 and 20% glutamine or glutamic acid,
1.6 to 3% arginine,
1.2 to 3% milk calcium,
0.2 to 1% EPA,
0.8 to 2% metformin,
1 to 2 mg of vitamin B6 per 30 g of composition without vehicles,
1 to 3 μg of vitamin D per 30 g of composition without vehicles,
75 to 150 μg of vitamin B9 per 30 g of composition without vehicles,
5 to 15 mg of vitamin E per 30 g of composition without vehicles,
   with the percentages being given by weight of dry material of all of the active ingredients that are present in the composition (except for possible vehicles), a portion of the components originating from the hydrolyzate, alpha-lactalbumin, the activator(s) of the cycles of AMPK and/or MAPK and/or PPARα and γ, and/or SIRT1, the regulator(s) of the cycles of AMPK and/or MAPK and/or PPARα and γ and/or SIRT1, and milk calcium, and the remainder being added freely in the form of vitamins and minerals and fatty acids.

The branched amino acids of the variants of compositions above consist of leukine, isoleukine, and valine, preferably:
7 to 10% leukine,
4 to 8% isoleukine, and
3 to 6% valine,
   and the aromatic amino acids of tryptophan, phenylalanine, and tyrosine preferably:
1 to 3% tryptophan,
2 to 6% phenylalanine, and
2 to 4% tyrosine.

The composition according to the invention can be obtained by a process as described below:
Obtaining a first mixture by mixing components in the following order:

lactoserum hydrolyzate, alpha-lactalbumin, and milk calcium; the pH should be around 7 and stabilized at this level,
Adding to the first mixture regulator(s) of the cycles of AMPK and/or MAPK and/or PPARα and γ and/or SIRT1 (nutritional regulator(s) and optionally regulating pharmaceutical active ingredient(s), and optionally vitamins, minerals, and supplementary fatty acids.

Vehicles and conventional feedstocks that are known to one skilled in the art can be added to the composition.

A powder that can be converted into a tablet or liquid is thus obtained, or else it is used in its powder form in packets, sticks, drums or capsules, for example. Preferably, it comes in the form of powder or granules packaged in a packet to be diluted in water. It can also come in the form of a ready-to-use drink, food bars, or logs.

The composition according to the invention, when it is administered orally in an adequate quantity, makes it possible in particular:
To prevent cardiometabolic risk
To prevent hepatic steatosis
To treat and/or to prevent prediabetes or diabetes
To treat and/or to prevent cardiometabolic diseases
To treat and/or to prevent infertility and/or subfertility of overweight men and women.

The various components of the composition according to the invention make it possible in particular to increase the secretion of adiponectin and serotonin, to decrease the inflammatory adipocytokines and insulin resistance for an action on the loss of visceral fat, hepatic fat, and deep subcutaneous fat.

Advantageously, it acts as an appetite suppressant and provides a feeling of fullness owing to an increase in the serotonin level and by the action of branched amino acids and milk calcium.

The composition according to the invention also makes it possible to permanently lose visceral fat in particular owing to:
The increase in the serotonin level in particular by the supply of tryptophan, which regulates fullness, directly on the cerebral level or indirectly in the intestine by the modulation of the secretion of GLP1 and CCK
The increase of the adiponectin level
The reduction of inflammation by a regulation of the signal NFkB of the macrophages.

In addition, it can slim the waistline while preserving lean body mass in particular by the supply of branched amino acids.

The composition according to the invention is also capable of acting on numerous other factors of cardiometabolic risk. In particular, it is capable of reducing stress, of normalizing arterial tension, of decreasing the inflammation of IL6, TNFα, PAI-1 and ultrasensitive CRP, of limiting coagulation (fibrinogen), of regulating cholesterol and triglycerides, of reducing glycemia and postprandial glycemia, insulinemia, and in a more general manner of regulating the cycles of the energy of the cell by the regulation of the APM/TP ratio through the cycles of AMPK, MAPK, PPARα and γ, and SIRT1.

The object of the invention is therefore the composition as described above for its application as a health product, in particular as a medication or as a medical nutrition product with oral administration for the prevention of cardiometabolic risk and/or for the prevention of hepatic steatosis and/or for the prevention and/or the treatment of type 2 prediabetes (and thus combating the development of type 2 diabetes), type 2 diabetes, cardiometabolic diseases and/or infertility and/or subfertility in humans.

In particular, the composition according to the invention can be used as a health product for decreasing the visceral fat and the deep subcutaneous fat in overweight persons.

The health product can be used specifically for reducing visceral fat and deep subcutaneous fat in overweight persons and/or for slimming the waistline and/or reducing stress and/or normalizing arterial tension and/or limiting oxidation and/or decreasing ultrasensitive CRP inflammation and/or limiting coagulation and/or regulating cholesterol and triglycerides and/or decreasing glycemia and postprandial glycemia and/or insulinemia and/or decreasing hypersensitivity of the intestinal barrier and/or rebalancing the intestinal flora and/or combating type 2 diabetes.

The daily dose of composition according to the invention (dose of a mixture of active ingredients without the vehicles) is preferably between 40 and 80 g, even more preferably in two servings of between 20 and 40 g taken at a point in time (in general 1 hour) before lunch and the other before dinner.

Advantageously, the bioavailability in the body of the amino acids, peptides and proteins that are present in the composition is between 10 and 30 minutes, which makes possible a quick action for initiating the feeling of fullness in such a way as to limit the quantity of food to be taken during the next meal.

In addition, the presence of milk calcium along with alpha-lactalbumin make it possible to enhance the palatability of the dietary product according to the invention by masking in particular the bitter taste of the lactoserum hydrolyzate in such a way that they work together to eliminate the risk that the persons will cease to consume it for reasons of taste and will abandon their regimen prematurely.

The combination of fatty acids, in particular EPA, with milk calcium and the particular mixture of amino acids according to the invention and released biopeptides also makes it possible to slow down the conversion of preadipocytes into visceral adipocytes by regulating MCP-1 (“Monocyte-chemoattracting protein-1”) to limit the action of macrophages of visceral adipose tissue and to accelerate the lipolysis of visceral adipocytes.

The invention also makes it possible:
To prevent the formation and the breakdown of atheromatous plaque in particular by significantly decreasing the secretion of inflammatory adipocytokines in such a way as to prevent cardiovascular accidents,
To limit the diabetic risk, in particular by decreasing the insulin resistance that is reflected by the reduction of insulinemia and of HOMA β, HOMA S, and HOMA-IR and by lowering glycemia by a better use of glucose in the muscles,
To combat chronic stress both at the level of the adaptation of a person to stress and the control of cortisol.

The composition can therefore be used as a health product with oral administration, acting:
By regulating the β-oxidation of the lipids, and/or
By increasing the use of glucose by the body, and/or
By decreasing insulin resistance and/or
By reducing the hyperpermeability of the intestinal barrier, and/or
By slowing down the conversion of preadipocytes into visceral adipocytes by regulation of MCP-1, and/or
By regulating the action of macrophages of visceral adipose tissue, and/or
By regulating the inflammation of adipose tissues, and/or
By accelerating the lipolysis of visceral adipocytes, and/or
By preventing the formation and breakdown of atheromatous plaque, and/or
By facilitating the exchanges of glucose and cholesterol and by enhancing the functioning of receptors of leptin, insulin and triglycerides, and/or
By initiating the effect of fullness.

The composition according to the invention, in particular the variant that contains metformin and/or statins, can be used in particular as a medication for the treatment of the metabolic syndrome and the prevention and/or the treatment of cardiometabolic diseases. Actually, such a composition makes it possible to treat the metabolic syndrome by decreasing the visceral fat, the hepatic fat, and the deep subcutaneous fat by making possible in particular an increase in the level of adiponectin and serotonin. In particular, the combined presence of at least one pharmaceutical active ingredient activating cycles of AMPK, MAPK, PPARα and γ and/or SIRT1 and at least one nutritional regulator of cycles of AMPK, MAPK, PPARα and γ and/or SIRT1 makes it possible to activate AMPK, MAPK, PPARα and γ and/or SIRT1 and therefore to increase the level of adiponectin.

The composition according to the invention can be used as a health product, in particular as a medication for prevention and primary, secondary and tertiary treatment of cardiometabolic diseases. In particular, it can be used for combating the factors of cardiometabolic risks, but also cardiometabolic diseases, in particular:
Cardiovascular diseases such as hypertension, arteritis of the lower limbs, coronary diseases, the sequelae of infarction or cerebral ictus,
Hyperglycemia and insulin resistance,
Type 2 prediabetes,
Type 2 diabetes,
Hyperlipidemia, and in particular the hyperlipidemia of type 2 diabetes,
Hepatic steatosis,
Metabolic subfertility and/or infertility of men and women.

The composition variant that contains metformin and/or one or more statin(s) makes it possible to create synergy between the pharmacological effects of the pharmaceutical active ingredient(s) and the same effects of nutritional active ingredients such as amino acids and small active biopeptides contained in the lactoserum hydrolyzate and alpha-lactalbumin as well as the nutritional regulators of the cycles of AMPK and/or MAPK and/or PPARα and PPARγ and/or SIRT1, and milk calcium. The composition according to the invention thus makes it possible to preserve and even to increase the effectiveness of the treatment by decreasing the doses of the pharmaceutical active ingredient(s) so as to minimize or cancel out their undesirable side effects.

Actually, the pharmaceutical active ingredients activating cycles of AMPK, MAPK, PPARα and γ, and/or SIRT1, such as metformin, are known for increasing the level of adiponectin, but at doses that bring about significant undesirable effects, in particular muscular myalgia for statins and manifestations that go from simple stomach upset to lactic acidosis, which can be fatal, by transitioning from nausea and vomiting, for metformin.

The composition according to the invention, in particular the variant that contains metformin and/or one or more statin(s), also makes it possible to enhance fertility in overweight men and women, in particular in persons who exhibit a cardiometabolic risk, by increasing in particular the level of adiponectin, decreasing the inflammatory adipocytokines, and the insulin resistance that are the factors that initiate this infertility and/or subfertility, in particular the syndrome of polycystic ovaries in women and the quantity and quality of sperm in men.

The composition makes possible the treatment of infertility and subfertility in general, in particular infertility or subfertility in men or women who present with a reduction in the adiponectin level thereof and/or an increase in inflammatory adipocytokines and insulinemia.

The object of the invention is specifically the use of the composition as medication for the treatment of infertility or subfertility in men or women who present with:
A waistline outside of the IDF 2005 standards (80/94 cm), and/or
Insulin resistance or type 2 diabetes, and/or
Dyslipidemia, and/or
Low-grade chronic inflammation, and/or
Hepatic steatosis, and/or
A state of anxiety or depression.

The composition can be used in particular:
In women:
   To combat the syndrome of polycystic ovaries and/or oligomenorrhea and/or amenorrhea and/or hyperandrogenism,
   To make possible a resumption of ovulation and/or to enhance folliculogenesis and/or to prevent miscarriages.
In men:
   To combat erectile dysfunction,
   To enhance oligozoospermia and/or asthenozoospermia,
   To enhance the quantity and the mobility of sperm cells.

The composition according to the invention is particularly suitable for the treatment of metabolic infertility or metabolic subfertility.

In terms of the invention, metabolic infertility or subfertility is defined as infertility or subfertility whose cause is a metabolic impairment that is reflected by a metabolic syndrome, in particular a waistline above international standards and/or insulin resistance or type 2 diabetes and/or dyslipidemia and/or hepatic steatosis and/or a low-grade chronic inflammation and/or a state of anxiety or depression. This metabolic infertility is reflected clinically by polycystic ovaries, endometriosis, anovulation, dysovulation, oligozoospermia, asthenozoospermia or erectile dysfunction.

The different components of the composition act in synergy for a very effective action on the enhancement of fertility in men or women, in particular in men or women who present with a metabolic syndrome.

The composition according to the invention is in particular capable of acting on adiponectin, inflammation, and insulinemia. However, the level of adiponectin is in general very low in infertile persons whereas the levels of us-CRP and TNFα are high or a level of insulinemia is high, in particular in persons with polycystic ovaries, endometriosis, oligozoospermia, asthenozoospermia or erectile dysfunction. Actually, the disruption of the levels of adiponectin or its mechanism of action produced by inflammation is associated with a disruption of fertility and fecundity. Actually, the regulation of adipocytokines is essential to preserving the integrity of the hypothalamic-hypophyseal-ovarian axis, to ovulation, to the success of nidation, and the progress of the pregnancy. Adiponectin is linked positively with SHBG and negatively with testosterone and TNFα. In addition, the reproductive system is coupled with the energy equilibrium. Thus, AMPK and PPARα and γ play a prominent role in being involved in the regulation of ovarian functioning in women, because they regulate the steroidogenesis of cells of the granulosa and ovocytic maturation. Certain hormones involved in metabolism use AMPK as a signal molecule for transmitting their ovarian and central effects to the reproduction function. An imbalance in this system is at the origin of pathologies such as the syndrome of polycystic ovaries and endometriosis.

The increase in the adiponectin level and the reduction of inflammation and insulin resistance makes possible, via the set of cycles of AMPK, MAPK, PPAR and SIRT1:
In women, a regulation of ovulation or a better response to treatments for stimulation of ovulation,
In men, an enhancement of erectile function or an increase in the quantity and a better mobility of sperm cells,
Action on testosterone: a decrease in women with polycystic ovaries and an increase in men of metabolic infertility or subfertility.

The increase in the adiponectin level and the reduction of inflammation and insulin resistance thus advantageously make it possible:
To treat polycystic ovaries, anovulation, and dysovulation through the increase in the phosphorylation of AMPK,
To treat endometriosis through MAPK,
To treat oligozoospermia and asthenozoospermia by an action on the testicles through PPARγ,
To treat erectile dysfunction through PPARγ and NO.

Furthermore, the composition according to the invention is capable of acting on the serotonin level. However, women that present with a syndrome of polycystic ovaries also have a low serotonin level. The men with infertility because of a metabolic syndrome or diabetes or who have continuous erectile dysfunction may have a low serotonin level of less than 80 ng/ml, responsible for oligospermy.

The presence optionally of selenium and zinc acts directly on the activation of AMPK and improves the insufficient testosterone level of 6,000 ng/l for men and 200 ng/l for women.

Finally, the composition according to the invention, in particular the composition variant that comprises metformin and/or one or more statin(s), can be used specifically as a health product, in particular as a medication, for the prevention of atherosclerosis. Atherosclerosis is characterized by a deposition of lipids on the intima of arteries, forming a core that is filled with fibers from deposits of lipids, foam cells, and finally macrophages that secrete adipocytokines. The final phase is the calcification of atheromatous plaque. The origin of atherosclerosis is the same as that of the metabolic syndrome; it involves excess cholesterol, triglycerides, hypertension, abdominal obesity, type 2 diabetes, nicotine addiction, chronic stress, a sedentary lifestyle, and pollution, and finally, family history. There is therefore a great similarity between atherosclerosis and the metabolic syndrome, although the consequence of atherosclerosis can be a single dyslipidemia linked to inflammation.

The adiponectin level is very low, and the levels of inflammation and insulinemia are high in persons suffering from atherosclerosis. Adiponectin is deficient in particular because of the increase in inflammatory adipocytokines, in particular TNFα, which act negatively through transcription, translation, and the turnover of AMPK, MAPK, PPAR and SIRT1, causing reduction thereof by dephosphorylation and therefore a decrease in the secretion of adiponectin.

The composition according to the invention makes it possible to increase the level of circulating adiponectin and/or to regulate its secretion over the long term.

Furthermore, in atherosclerosis, the composition according to the invention is capable of acting on the serotonin level.

The invention also makes it possible:
To prevent the formation and the breakdown of atheromatous plaque, in particular by significantly decreasing the secretion of inflammatory adipocytokines, in particular the us-CRP, in such a way as to prevent cardiovascular accidents,
To limit the diabetic risk, in particular by restoring the elasticity of the cellular membrane by matrix remodeling, which facilitates the exchanges of glucose and cholesterol and enhances the functioning of the receptors of leptin, insulin, and triglycerides, and
To combat chronic stress both at the level of the adaptation of the person to stress and the control of cortisol.

The composition according to the invention, regardless of its variant, can therefore be used as a health product for different therapeutic applications.

The composition that comprises an active ingredient mixture that consists of at least:
A lactoserum hydrolyzate with a molecular weight of between 200 and 5,000 daltons,
Alpha-lactalbumin,
At least one regulator of the cycles of AMPK and/or MAPK and/or PPARα and γ and/or SIRT1, selected from among the family of tocopherols, vitamin B6, leukine, and the family of branched amino acids, and
Milk calcium,
   can preferably be used for preventing cardiometabolic risk, preventing hepatic steatosis, and/or treating type 2 diabetes and prediabetes, in particular as a medical nutrition product.

The composition comprising an active ingredient mixture that consists of at least:
A lactoserum hydrolyzate with a molecular weight of between 200 and 5,000 daltons,
Alpha-lactalbumin,
At least one activator of the cycles of AMPK and/or MAPK and/or PPARα and γ and/or SIRT1, selected from among metformin and statins,
At least one regulator of the cycles of AMPK and/or MAPK and/or PPARα and γ and/or SIRT1, selected from among the family of tocopherols and/or vitamin B6, milk calcium,
   can be used preferably for preventing cardiometabolic risk, preventing and/or treating hepatic steatosis and/or preventing and/or treating type 2 diabetes and prediabetes, preventing and/or treating cardiovascular diseases, preventing and/or treating infertility or subfertility, preventing atherosclerosis, in particular as a medication.

The invention is now illustrated by nonlimiting examples of compositions that come in the form of a 40 g powder (active ingredients and vehicles) packaged in a packet, adapted for use as a health product.

### Example 1 of Composition and Use for the Prevention of Cardiometabolic Risk, Prediabetes, Hepatic Steatosis and Metabolic Infertility or Subfertility

This composition is obtained from the following active ingredients:
14.8 g of lactoserum hydrolyzate with a molecular weight of between 200 and 3,500 daltons, with a degree of hydrolysis of 25%, or 12 g of proteins/peptides of lactoserum,
13.3 g of alpha-lactalbumin, including 12 g of proteins/peptides,
1 mg of vitamin B6,
10 mg of vitamin E, preferably in the form of a tocopherol mixture,
100 μg of vitamin B9,
2.5 μg of vitamin D,
750 mg of omega-3 in the form of EPA,
20 mg of SOD,
25 mg of glutathione,
Enough for 0.28 g of milk calcium (total in the composition: 0.4 g).

In lactoserum and alpha-lactalbumin, this composition comprises:
2.6 g of leukine,
1.4 g of isoleukine,
4.3 g of glutamine,
0.5 g of tryptophan,
0.6 g of arginine.

When this medical nutrition product is administered for therapy twice daily (one packet before lunch and one packet before dinner) for at least 120 days to patients who present with signs associated with cardiometabolic risk, a slimming of the waistline on the order of 5% relative to the initial waistline is noted, which is characterized by a significant decrease in fat and in particular deep subcutaneous and visceral fat. An administration for 200 to 240 days makes possible a loss of 10% of the waistline relative to the initial waistline. The loss of body fat is usually more significant than the total weight loss, which involves a relative increase in the lean body mass since it is not lowered with the invention because of the composition and in particular branched amino acids and leukine. The composition according to the invention makes it possible in particular to decrease the visceral fat between 5 and 10%, according to the treatment time, of the total initial weight, with this decrease signifying a decrease in the risk of cardiometabolic diseases. It is reflected in particular by:
A decrease in the total cholesterol level,
A decrease in the LDL cholesterol level,
A rise in the HDL cholesterol level,
A decrease in triglycerides,
A decrease in fasting glycemia and in glycemia produced in patients with insulin resistance or glucose intolerance and HbA1c in patients suffering from type 2 diabetes,
A decrease in insulinemia, HOMAβ, HOMA S and HOMA-IR,
Normalization of diastolic and systolic arterial tension,
Normalization of us-CRP and fibrinogen, and
An increase in adipocytic and plasmatic adiponectin.

### Example 2 of Composition and Use for the Prevention/Treatment of Prediabetes, Diabetes, and Metabolic Infertility and Subfertility

This composition is obtained from the following active ingredients:
14.8 g of lactoserum hydrolyzate with a molecular weight of between 200 and 3,500 daltons, with a degree of hydrolysis of 25%, or 12 g of proteins/peptides of lactoserum,
13.3 g of alpha-lactalbumin, including 12 g of proteins/peptides,
250 mg of metformin hydrochloride,
10 mg of vitamin E, preferably in the form of a tocopherol mixture,
1 mg of vitamin B6,
100 μg of vitamin B9,
2.5 μg of vitamin D,
Enough for 280 mg of milk calcium (total in the composition: 0.4 g),
25 μg of selenium,
7.5 mg of zinc,
750 mg of omega-3 in the form of EPA,
20 mg of SOD,
25 mg of glutathione.

This composition comprises:
2.6 g of leukine,
1.4 g of isoleukine,
4.3 g of glutamine,
0.5 g of tryptophan,
0.6 g of arginine.

### Application of Composition 2 for Prediabetes and Diabetes:

The composition makes it possible in particular to decrease visceral fat between 5 and 10%, according to the treatment time, of the total initial weight, with this decrease signifying a decrease in the risk of diabetic diseases. It is reflected in particular by:
A decrease in fasting glycemia and in glycemia produced in patients with insulin resistance or glucose intolerance and HbA1c in patients suffering from type 2 diabetes,
A decrease in insulinemia, HOMAβ, HOMA S and HOMA-IR,
Normalization of us-CRP and fibrinogen, and
An increase in plasmatic serotonin,
An increase in adipocytic and plasmatic adiponectin.
   Application of Composition 2 for the Prevention and/or Treatment of Infertility or Subfertility:

When the composition of Example 2 is administered for therapy twice daily (one packet before lunch and one packet before dinner) for at least 120 days to patients who present with signs associated with cardiometabolic risk, in particular a slimming of the waistline on the order of 5% relative to the initial waistline is noted, which is characterized by a significant decrease in fat and in particular deep subcutaneous and visceral fat. An administration for 200 to 240 days makes possible a loss of 10% of the waistline relative to the initial waistline. The loss of body fat is usually more significant than the total weight loss, which involves a relative increase of the lean body mass. The composition according to the invention makes it possible in particular to decrease the visceral fat between 5 and 10% (according to the treatment time) of the total initial weight, with this decrease signifying a decrease in the risk of subfertility and infertility, in particular of metabolic subfertility and infertility, and cardiometabolic diseases. It is reflected in particular by:
A decrease in the free testosterone of women with polycystic ovaries, and/or
An increase in progesterone, and/or
A decrease in LH (gonadotrophin), and/or
An elimination of inflammation in the endometrium, and/or
A restoration of erectile function with an enhancement of the level of testosterone and nitric oxide NO, and/or
A decrease in the total cholesterol level, and/or
A decrease in the LDL cholesterol level, and/or
A stabilization in the reduction of the HDL cholesterol level, and/or
A decrease in triglycerides, and/or
A decrease in fasting glycemia and in glycemia produced in persons with insulin resistance or glucose intolerance, and/or
A decrease in HbA1c in persons suffering from type 2 diabetes, and/or
A decrease in insulinemia, and/or
Normalization of ultrasensitive CRP and fibrinogen.

### Example 3 of a Composition and Its Use for the Primary, Secondary, and Tertiary Prevention and the Treatment of Cardiovascular Diseases and Atherosclerosis

This composition is obtained from the following active ingredients:
14.8 g of lactoserum hydrolyzate with a molecular weight of between 200 and 3,500 daltons, with a degree of hydrolysis of 25%, or 12 g of proteins/peptides of lactoserum,
13.3 g of alpha-lactalbumin, including 12 g of proteins/peptides,
5 mg of simvastatin,
10 mg of vitamin E, preferably in the form of a tocopherol mixture,
1 mg of vitamin B6,
100 μg of vitamin B9,
2.5 μg of vitamin D,
Enough for 280 mg of milk calcium (total in the composition: 0.4 g),
25 μg of selenium,
7.5 mg of zinc,
750 mg of omega-3 in the form of purified EPA,
20 mg of SOD,
25 mg of glutathione.

This composition comprises:
2.6 g of leukine,
1.4 g of isoleukine,
4.3 g of glutamine,
0.5 g of tryptophan,
0.6 g of arginine.

### Application of Composition 3 for the Prevention and Treatment of Cardiovascular Diseases

When this composition is administered for therapy twice daily (one packet before lunch and one packet before dinner) for at least 120 days to patients who present with signs associated with cardiovascular risk, a slimming of the waistline on the order of 5% relative to the initial waistline is noted, which is characterized by a significant decrease in fat and in particular deep subcutaneous and visceral fat. An administration for 200 to 240 days makes possible a loss of 10% of the waistline relative to the initial waistline. The loss of body fat is usually more significant than the total weight loss, which involves a relative increase in the lean body mass since it is not lowered with the invention because of the composition and in particular branched amino acids and leukine. The composition according to the invention makes it possible in particular to decrease the visceral fat between 5 and 10%, according to the treatment time, of the total initial weight, with this decrease signifying a decrease in the risk of cardiovascular diseases. It is reflected in particular by:
A decrease in the total cholesterol level,
A decrease in the LDL cholesterol level,
A rise in the HDL cholesterol level,
A decrease in triglycerides,
Normalization of diastolic and systolic arterial tension,
Normalization of TNFα, IL6 and us-CRP and fibrinogen, and
An increase in adipocytic and plasmatic adiponectin.

### Application of Composition 3 for the Prevention of Atherosclerosis

When the composition of Example 2 is administered for therapy twice daily (one packet before lunch and one packet before dinner) for at least 120 days to patients who present with signs associated with atherosclerosis and/or impairment of metabolic aging, a slimming of the waistline on the order of 5% relative to the initial waistline is noted, which is characterized by a significant decrease in fat and in particular deep subcutaneous and visceral fat. An administration for 200 to 240 days makes possible a loss of 10% of the waistline relative to the initial waistline. The loss of body fat is usually more significant than the total weight loss, which involves a relative increase of the lean body mass. The composition according to the invention makes it possible in particular to decrease the visceral fat between 5 and 10%, according to the treatment time, of the total initial weight, with this decrease signifying a decrease in atherosclerosis. It is reflected in particular by:
A decrease in the total cholesterol level,
A decrease in the LDL cholesterol level,
An increase in the HDL cholesterol level,
A decrease in triglycerides,
A decrease in insulin resistance,
Normalization of diastolic and systolic arterial tension,
Enhancement of the factors of homeostasis such as fibrinogen,
Matrix remodeling with enhancement of membrane elasticity,
Normalization of us-CRP and adipocytic and plasmatic markers of inflammation,
Increase in plasmatic and adipocytic adiponectin,
Increase in serotonin.
   Example 4 of Composition and Use for the Treatment of Cardiovascular Diseases with Type 2 Diabetes or Diabetes with a Cardiovascular Risk

This composition is obtained from the following active ingredients:
14.8 g of lactoserum hydrolyzate with a molecular weight of between 200 and 3,500 daltons, with a degree of hydrolysis of 25%, or 12 g of proteins/peptides of lactoserum,
13.3 g of alpha-lactalbumin, including 12 g of proteins/peptides,
2.5 mg of atorvastatin,
10 mg of vitamin E, preferably in the form of a tocopherol mixture,
1 mg of vitamin B6,
100 μg of vitamin B9,
2.5 μg of vitamin D,
Enough for 280 mg of milk calcium (total in the composition: 0.4 g),
25 μg of selenium,
7.5 mg of zinc,
750 mg of omega-3 in the form of purified EPA,
20 mg of SOD,
25 mg of glutathione.

This composition comprises:
2.6 g of leukine,
1.4 g of isoleukine,
4.3 g of glutamine,
0.5 g of tryptophan,
0.6 g of arginine.
   Application in Cardiovascular Diseases with Diabetes or Diabetes with a Cardiovascular Risk in Particular of High Triglycerides

When the composition of Example 2 is administered for therapy twice daily (one packet before lunch and one packet before dinner) for at least 120 days to patients who present with signs associated with atherosclerosis and/or impairment of metabolic aging, a slimming of the waistline on the order of 5% relative to the initial waistline is noted, which is characterized by a significant decrease in fat and in particular in deep subcutaneous and visceral fat. An administration for 200 to 240 days makes possible a loss of 10% of the waistline relative to the initial waistline. The loss of body fat is usually more significant than the total weight loss, which involves a relative increase of the lean body mass. The composition according to the invention makes it possible in particular to decrease the visceral fat between 5 and 10%, according to the treatment time, of the total initial weight, with this decrease signifying a decrease in risks linked to diabetes primarily associated with high triglycerides, which, according to prevalent studies, are the most dangerous associations for a cardiovascular accident. It is reflected in particular by:
A decrease in the total cholesterol level,
A decrease in the LDL cholesterol level,
An increase in the HDL cholesterol level,
A decrease in triglycerides,
A decrease in fasting glycemia and in glycemia produced in patients with insulin resistance or glucose intolerance and HbA1c in patients suffering from type 2 diabetes,
A decrease in insulinemia, HOMAβ, HOMA S and HOMA-IR,
Normalization of arterial tension,
Enhancement of the factors of homeostasis such as fibrinogen,
Matrix remodeling with enhancement of membrane elasticity,
Normalization of us-CRP and adipocytic and plasmatic markers of inflammation,
Increase in plasmatic and adipocytic adiponectin,
An increase in plasmatic serotonin.

The use of the composition according to the invention, regardless of its variant, as a health product is accompanied by a change in lifestyle for further enhancing its effects. Actually, in chronic diseases, the problem is more complicated than in the case of an acute disease where in general all that needs to be done is to apply the diagnosis, formulate the treatment, inform the patient about it, and follow, through analyses, the results of the treatment, and adapt it if necessary.

The composition according to the invention is therefore preferably used as a health product to supplement a change in lifestyle making it possible to support the treatment within the framework of the increase in adiponectin and serotonin, the decrease of insulinemia and of inflammation in particular.

The composition according to the invention is preferably used as a health product to supplement:
A particular diet,
An appropriate limiting of calorie intake, and/or
Management of chronic stress, and/or
Management of a tendency toward anxiety or depression, and/or
Physical activity and the fight against a sedentary lifestyle.

The diet is to include in particular a consumption of carbohydrates with low glycemic feedstocks (or a low glycemic index) of in general less than 20 and/or a consumption of soluble and insoluble fibers and/or an elimination of saturated fatty acids and the partial replacement of these saturated fatty acids by polyunsaturated fatty acids and/or a consumption of omega-3 fatty acids, and/or the elimination of glycation products (by Maillard reaction).

The objective of such a diet is to limit postprandial glycemia and related inflammation peaks, source of cardiovascular accidents, to limit excessive oxidation of carbohydrates, to lower the inflammation of adipocytokines, and to cause the conversion of preadipocytes into adipocytes and the infiltration of macrophages and finally the lipolysis of visceral adipocytes to stop.

The drastic limiting of the glycation products makes it possible to reduce inflammation; this requires an often radical change in cooking methods, in particular fried foods.

The supply of omega-3 and non-fatty animal proteins of fish and white meat also makes it possible to promote lipolysis, in particular on the hepatic level. In addition, it significantly increases adiponectin.

Furthermore, a moderate consumption of red wine (1 glass daily) makes it possible to enhance the increase in the adiponectin level.

Relative to limiting calorie intake, it makes it possible to increase SIRT1 and therefore acts to promote an increase in the adiponectin level. It is necessary, however, to ensure that the diet is not too restrictive, in particular greatly below the basic energy expenditure, because the reaction on the cellular level will be violent by blocking the AMP/ATP ratio through AMPK and PPARγ and will bring about a rebound when the limiting of calorie intake is stopped.

Management of chronic stress is preferably carried out by holistic methods or else general or specific physical activities for lowering the circulating cortisol. This management of chronic stress makes it possible to have an impact on AMPK, MAPK, PPARγ and SIRT 1.

Management of the tendency toward anxiety or depression can be carried out by holistic methods or else general or specific physical activities. The supply of tryptophan and therefore of serotonin makes it possible to improve the background of anxiety or depression that is common to numerous overweight persons. Likewise, the quality of sleep is enhanced by the production of cerebral serotonin. Finally, by loss of weight and body fat, sleep apnea often disappears.

Finally, physical activity, and the fight against the sedentary lifestyle, is an important factor, which makes it possible in particular to increase AMPK and SIRT1 and therefore adiponectin over the long term. Physical activity should be moderate and last at least 30 minutes. In contrast, intense and prolonged activities (such as marathons) are a source of high oxidation.

Limiting of calorie intake and the increase in physical activity act on the cycles of AMPK, PPAR and SIRT1 in a significant manner, and conversely, the biochemical increase of AMPK, PPAR, and SIRT1 mimics the action of the limiting of calorie intake and physical activity. This is why the combination of a composition that is capable of increasing in particular AMPK, PPAR, and SIRT1 and a limiting of calories associated with physical activity has an impact that is extremely favorable on the prevention of the factors of cardiometabolic risk, hepatic steatosis, and the treatment of prediabetes.

This use of the composition to which a change in lifestyle is added (adaptation of diet and/or limiting of calorie intake and/or increase in physical activity and the fight against the sedentary lifestyle and/or management of stress and/or management of states of anxiety or depression) requires the patient's taking charge of himself, which requires in particular therapeutic education of the patient.

According to a suitable variant of the invention, the use of the health product composition is accompanied by a process that comprises in particular:
The determination of an objective for slimming the waistline,
The training of the patient to make a change in lifestyle.

The therapeutic education of the patient makes two conditions necessary:
The motivation of the patient,
The training of the patient with therapeutic education.

The motivation of the patient within the framework of health is difficult to implement when it involves a change in lifestyle, primarily if a change in more than 20% of the habits that are anchored in the personality of the patient (family, region, religion, social background, professional background, etc.) is targeted.

In a preferred way, the use of the composition as a health product is also accompanied by the determination of an objective of slimming the waistline.

The motivation of the patient through an objective of slimming the waistline provides him with a simple objective in his search for prevention or treatment.

Relative to the training of the patient with therapeutic education, the difficulty resides in the number of persons involved, since the metabolic syndrome concerns 25 to 35% of the adult population or millions of persons. However, the paramedical and medical structures are not capable of forming such a mass of the population. It is therefore preferred to have recourse to e-health in a personalized way but also standardized by phenotypes because the contact is to be frequent, ideally daily.

Preferably, the use of the composition as a health product is also accompanied by training the patient to a change in lifestyle by example and by the action of training.

In particular, the use of the composition as a health product can be accompanied by therapeutic teaching of e-health, in particular by the use of a medical video game in which an avatar of the patient evolves as his model. The patient gives information about his medical data and development thereof, as well as information relative to taking the health product, his diet, his limiting of calorie intake, his chronic stress, his tendency toward depression, and his pharmacological medication if necessary. The avatar reacts like the patient who will then receive messages based on his negative or positive development. Each development of the avatar is explained with teaching on the negative or positive points.

The object of the invention is therefore also the use of the composition as a health product accompanied by a process for therapeutic education that comprises in particular:
The motivation of the patient by the determination of an objective of slimming the waistline, and/or
The training of the patient to a change in lifestyle by the creation of his avatar in a medicalized video game who reacts as he would and from which teaching must be drawn.

This makes it possible also to enhance the effectiveness of the composition according to the invention.

1. A composition, suitable for use as a health product with oral administration, comprising a mixture of active ingredients that comprise at least:
a lactoserum hydrolyzate with a molecular weight of between 200 and 5,000 daltons,
alpha-lactalbumin,
at least one regulator of AMPK, MAPK, PPARα and γ and/or SIRT1 cycles, selected from the group consisting of tocopherols, vitamin B6, leukine, and branched acids, and
milk calcium.

2. The composition according to claim 1, wherein 55% of the total of molecules of the lactoserum hydrolyzate has a molecular weight that is less than or equal to 1,000 daltons.

3. The composition according to claim 1, wherein the lactoserum hydrolyzate has a degree of hydrolysis of between 15% and 35%.

4. The composition according to claim 1, wherein the ratio by weight of proteins between alpha-lactalbumin and the lactoserum hydrolyzate is between 50/50 and 30/70.

5. The composition according to claim 1, wherein the quantity of the at least one regulator of the AMPK, MAPK, PPARα and γ and/or SIRT1 cycles is 5 to 30 mg for between 25 and 40 g of composition.

6. The composition according to claim 1, wherein the composition comprises at least one amino acid selected from the group consisting of tryptophan, glutamine, leukine, isoleukine, and arginine, with the at least one amino acid being obtained from components of the active ingredient mixture.

7. The composition according to claim 6, wherein the tryptophan represents between 6 and 9% by weight of neutral amino acids that are present in the composition.

8. The composition according to claim 1, wherein the active ingredient mixture further comprises at least one freely-added selected from the group consisting of vitamin B9 and vitamin D.

9. The composition according to claim 1, wherein the active ingredient mixture further comprises essential fatty acids.

10. The composition according to claim 9, wherein the essential fatty acids are omega-3 fatty acids of vegetable origin ALA, with a high proportion of EPA or purified EPA.

11. The composition according to claim 1, wherein the lactoserum hydrolyzate represents between 40 and 60%, the alpha-lactalbumin represents between 38 and 55%, and the milk calcium represents between 1 and 3%, with the percentages being given by weight of dry material of all of the active ingredients that are present in the composition.

12. The composition according to claim 1, comprising at least:
1 to 3% trytophan, by weight of dry material of all of the active ingredients that are present in the composition,
12 to 20% branched amino acids, by weight of dry material of all of the active ingredients that are present in the composition,
6 to 10% aromatic amino acids, by weight of dry material of all of the active ingredients that are present in the composition,
10 and 20% glutamine or glutamic acid, by weight of dry material of all of the active ingredients that are present in the composition,
1.6 to 3% arginine, by weight of dry material of all of the active ingredients that are present in the composition,
1.2 to 3% milk calcium, 0.2 to 1% EPA, by weight of dry material of all of the active ingredients that are present in the composition,
1 to 2 mg of vitamin B6 per 30 g of composition without vehicles,
1 to 3 μg of vitamin D per 30 g of composition without vehicles,
75 to 150 μg of vitamin B9 per 30 g of composition without vehicles, and
5 to 15 mg of vitamin E per 30 g of composition without vehicles.

13. The composition according to claim 1, wherein the composition is in a form selected from the group consisting of powder, granules, tablets, capsules, and liquid.

14. The composition according to claim 1, further comprises at least one pharmaceutical active ingredient that regulates AMPK, MAPK, PPARα and γ and/or SIRT1 cycles is selected from from the group consisting of metformin and statins.

15. The composition according to claim 14, wherein the at least one pharmaceutical active ingredient that regulates the AMPK, MAPK, PPARα and γ SIRT1 cycles is a statin selected from the group consisting of simvastatin, pravastatin, rosuvastatin, atorvastatin, fluvastatin, lovastatin, and combinations thereof.

16. The composition according to claims 14, wherein the quantity of the at least one pharmaceutical active ingredient that regulates the AMPK, MAPK, PPARα and γ and/or SIRT1 cycles is 200 to 700 mg for metformin and between 2.5 to 10 mg for statins, and between 20 and 40 g of composition without vehicles.

17. The composition according to claim 1, comprising at least:
1 to 3% tryptophan, by weight of dry material of all of the active ingredients that are present in the composition,
12 to 20% branched amino acids, by weight of dry material of all of the active ingredients that are present in the composition,
6 to 10% aromatic amino acids, by weight of dry material of all of the active ingredients that are present in the composition,
10 and 20% glutamine or glutamic acid, by weight of dry material of all of the active ingredients that are present in the composition,
1.6 to 3% arginine, by weight of dry material of all of the active ingredients that are present in the composition,
1.2 to 3% milk calcium, by weight of dry material of all of the active ingredients that are present in the composition,
at least one compound selected from the group consisting of 200 to 600 mg of metformin, 5 to 20 mg of simvastatin, and 2.5 to 5 mg of atorvastatin, the at least one compound mass being per 20 to 40 g of the composition without vehicles,
0.2 to 1% EPA,
1 to 2 mg of vitamin B6 per 30 g of the composition without vehicles,
1 to 3 μg of vitamin D per 30 g of the composition without vehicles,
75 to 150 μg of vitamin B9 per 30 g of the composition without vehicles, and
5 to 15 mg of vitamin E per 30 g of the composition without vehicles.

18-19. (canceled)

20. A method of preventing hepatic steatosis and/or treating type 2 prediabetes, comprising orally administering to a subject in need thereof an effective amount of the composition according to claim 1.

21. The method according to claim 20, wherein the method treats type 2 diabetes.

22. A method of treating infertility and/or subfertility in overweight men or women, comprising orally administering to a subject in need thereof an effective amount of the composition according to claim 1.

23. The method according to claim 22, the subject has a reduction of adiponectin level and/or serotonin level.

24. The method according to claim 22, wherein the subject has a condition selected from the group consisting of: a waistline outside of the IDF 2005 standards, insulin resistance or type 2 diabetes, dyslipidemia, low-grade chronic inflammation, hepatic steatosis, a state of depression or in a state of depression, and combinations thereof.

25. The method according to claim 22, wherein the subject is a woman that has a syndrome selected from the group consisting of polycystic ovaries, oligomenorrhea, amenorrhea, hyperandrogenism and combinations there of or or the subject is man that has erectile dysfunction, oligozoospermia, asthenozoospermia, and combinations thereof.

26. A method of preventing and/or treating cardiometabolic diseases comprising orally administering to a subject in need thereof an effective amount of the composition according to claim 1.

27. A method of preventing atherosclerosis comprising orally administering to a subject in need thereof an effective amount of the composition according to claim 1.

28. A method of preventing and/or treating at least one of cardiovascular diseases hyperglycemia and at least one of insulin resistance hyperlipidemia comprising orally administering to a subject in need thereof an effective amount of the composition according to claim 1.

29. A method of preventing or treating hypertension, arteritis of the lower limbs, coronary diseases, and the sequelae of infarction or cerebral ictus comprising orally administering to a subject in need thereof an effective amount of the composition according to claim 1.

30. The method according to claim 22, wherein the oral administration of the effective amount of the composition supplements an activity selected from the group consisting of a limiting of calorie intake, chronic management of stress, physical activity and combinations thereof.

31. A method of preventing cardiometabolic risk comprising orally administering to a subject in need thereof an effective amount of the composition according to claim 1.

32. A method of treatment selected from the group consisting of decreasing visceral fat and deep subcutaneous fat in overweight persons, slimming the waistline, reducing stress, normalizing arterial tension, decreasing the ultrasensitive CRP inflammation, limiting coagulation, regulating cholesterol and triglycerides, decreasing glycemia and postprandial glycemia, insulinemia, decreasing the hypersensitivity of the intestinal barrier, the intestinal flora, remodeling the cellular matrix, and combinations thereof, said method comprising orally administering to a subject in need thereof an effective amount of the composition of claim 1.

33. The method according to claim 31, wherein the oral administration of the effective amount of the composition supplements an activity selected from the group consisting of a limiting of calorie intake, chronic management of stress, physical activity and combinations thereof.
